(19) United States
(12) Patent Application Publication
(10) Pub. No.: US 2025/0218553 A1
(43) Pub. Date: Jul. 3, 2025

(54) ASSESSMENT METHOD, PROGRAMM, COMPUTER-READABLE DATA-CARRIER, COMPUTING DEVICE, AND ARRANGEMENT FOR A COMPUTER ASSISTED SUBSTANCE ASSESSMENT

(71) Applicant: Airbus SAS, BLAGNAC (FR)

(72) Inventors: Camille CARRÉ, BLAGNAC (FR); Akshat TANDON, BLAGNAC (FR)

(21) Appl. No.: 18/987,520

(22) Filed: Dec. 19, 2024

(30) Foreign Application Priority Data
Dec. 28, 2023 (EP) 23220577.3

(51) Int. Cl. G16C 20/30; G16C 20/70

(52) U.S. Cl. CPC G16C 20/30; G16C 20/70

(57) ABSTRACT

An assessment method, a corresponding assessment program, a computer-readable data-carrier having stored thereon the assessment program, a computing device configured to carry out the assessment program and/or comprising the computer-readable data-carrier, and an assessment arrangement for a computer assisted assessment of at least one property parameter. Generally, the method includes obtaining a molecular structure of the chemical substance; obtaining at least one reference structure of at least one reference substance having at least one reference parameter, comparing the molecular structure to the at least one reference structure; and deriving the at least one property parameter from the comparison based on the at least one reference parameter.

### CROSS-REFERENCES TO RELATED APPLICATIONS

This application claims the benefit of European Patent Application Number 23220577.3 filed on Dec. 28, 2023, the entire disclosure of which is incorporated herein by way of reference.

### FIELD OF THE INVENTION

The present disclosure relates to the field of computational chemistry in substance development and assessment of their chemical and/or physical properties. In particular, the disclosure relates to an assessment method for computer assisted assessment of at least one property parameter of a chemical substance, to an assessment program, a computer-readable data-carrier, a computing device, and to an assessment arrangement for a computer assisted assessment of at least one property parameter of a chemical substance.

### BACKGROUND OF THE INVENTION

Methods for computer assisted assessment of molecule and material properties are known from the prior art. Such methods allow for predicting respective properties on a theoretical level. The prediction can help in at least a first assessment of a certain chemical and/or physical behavior of a substance. Based on the prediction, candidates of substances for comprehensive chemical and/or physical assessment through respective laboratory and/or field tests may be shortlisted, thus enabling to streamline respective assessment and testing procedures which can be extensively time and resource consuming.

Wang, Y., Wang, J., Cao, Z. et al., “Molecular contrastive learning of representations via graph neural networks”, Nat Mach Intell 4, 279-287 (2022). https://doi.org/10.1038/s42256-022-00447-x, for example, present MolCLR (Molecular Contrastive Learning of Representations via Graph Neural Networks), a self-supervised learning framework that leverages large unlabeled data (˜10 million unique molecules). In a MolCLR pre-training, molecule graphs are built, and graph-neural-network encoders are developed to learn differentiable representations. Three molecule graph augmentations are proposed: atom masking, bond deletion and subgraph removal. A contrastive estimator maximizes the agreement of augmentations from the same molecule while minimizing the agreement of different molecules. Experiments are supposed to show that such contrastive learning framework significantly improves the performance of graph-neural-network encoders on various molecular property benchmarks including both classification and regression tasks. MolCLR learns to embed molecules into representations that can distinguish chemically reasonable molecular similarities.

Wang Y, Magar R, Liang C, Barati Farimani A, “Improving Molecular Contrastive Learning via Faulty Negative Mitigation and Decomposed Fragment Contrast”, J Chem Inf Model. 2022 Jun. 13; 62 (11): 2713-2725. doi: 10.1021/acs.jcim.2c00495. Epub 2022 May 31. PMID: 35638560, state that so-called deep learning has been a prevalence in computational chemistry and widely implemented in molecular property predictions. Recently, self-supervised learning (SSL), especially contrastive learning (CL), has gathered growing attention for the potential to learn molecular representations that generalize to the gigantic chemical space. Unlike supervised learning, SSL can directly leverage large unlabeled data, which greatly reduces the effort to acquire molecular property labels through costly and time-consuming simulations or experiments. However, most molecular SSL methods borrow the insights from the machine learning community but neglect the unique cheminformatics (e.g., molecular fingerprints) and multilevel graphical structures (e.g., functional groups) of molecules. They propose iMolCLR as an improvement of Molecular Contrastive Learning of Representations with graph neural networks (GNNs) in two aspects: (1) mitigating faulty negative contrastive instances via considering cheminformatics similarities between molecule pairs and (2) fragment-level contrasting between intramolecular and intermolecular substructures decomposed from molecules.

Assessment methods, as known from the prior art, do not seem to satisfy all requirements for similarity and/or property predictions for chemical substances. Known methods commonly rely on augmentations. The implemented way of SSL may not achieve desired results in similarity and/or prediction accuracy.

### SUMMARY OF THE INVENTION

It may thus be seen as an object to provide alternative methods for similarity and/or property predictions for the assessment of chemical substances. In particular, it may be seen as an object to provide a way of implementing SSL for achieving a relatively high similarity and/or property prediction accuracy while maintaining computational and analytical efficiency. These objects are at least partly achieved by the subject-matter of the independent claims.

According to an aspect, an assessment method for computer assisted assessment of at least one property parameter of a chemical substance is provided, comprising the steps of obtaining a molecular structure of the chemical substance; obtaining at least one reference structure of at least one reference substance having at least one reference parameter, comparing the molecular structure to the at least one reference structure; and deriving the at least one property parameter from the comparison based on the at least one reference parameter.

An assessment program comprising instructions is provided which, when the program is executed by a computing device, cause the computing device to carry out a corresponding assessment method.

A computer-readable data-carrier is provided having stored thereon a corresponding assessment program.

A computing device is provided which is configured to carry out a corresponding assessment program and/or comprises a corresponding computer-readable data-carrier.

An assessment arrangement for a computer assisted assessment of at least one property parameter of a chemical substance is provided, configured to carry out a corresponding assessment method, comprising a corresponding assessment program, comprising a corresponding computer-readable data-carrier, and/or comprising a corresponding computing device.

Alternatively, or additionally, a training method, as well as corresponding training program may be provided, and maybe stored on a computer-readable data-carrier. A computing device may be configured to carry out the training program and/or may comprise respective computer-readable data-carrier. The assessment arrangement thus may comprise a training arrangement, and/or the training arrangement may be provided in addition to the assessment arrangement and may be configured to carry out the training method, may comprise the training program, a corresponding computer-readable data-carrier, and/or the corresponding computing device. The training method may comprise any of the steps of the assessment method as described herein, in particular any steps performed in connection with the prediction algorithm, training parameters, asset or training phases.

The assessment method itself does neither rely on augmentation to find similar molecules, nor on calculating a loss for a pair of structures to be compared to each other. The chemical substances may comprise, for example, halon and other extinguishing agents, PFAS, Bisphenols and other REACH-listed components from resin systems, corrosion inhibitors e.g., chromates, etc., as well as derivates and products of decomposition thereof. The at least one property parameter may represent possible and/or probable toxicity, density, Young's modulus, Tensile strength, boiling points, viscosity, flammability, fuel ignition, corrosivity, optical behavior, and/or magneticity of the chemical substance.

The proposed solution has the advantage over the prior art, that for each comparing step, a direct comparison between the molecular structure and the reference structure can be carried out. Consequently, a triangular relationship and/or similarity analysis between a molecule structure, and our augmented version thereof, and a reference structure of a reference substance to be compared therewith is not necessary. Any augmentation step of (randomly) altering molecules and respective computational and analytic efforts can be omitted. Thus, a high similarity and/or property prediction accuracy can be achieved with high computational and analytical efficiency.

Further developments can be derived from the dependent claims and from the following description. Features described with reference to devices and arrangements may be implemented as method steps, or vice versa. Therefore, the description provided in the context of the computing device and/or assessment arrangement applies in an analogous manner also to respective methods. In particular, the functions of the computing device and/or of the assessment arrangement and of their or its, respectively, components may be implemented as method steps of the methods and the method steps may be implemented as functions of the computing device and/or of the assessment arrangement.

According to a possible embodiment of an assessment method, the step of comparing involves identifying a similarity between the molecular structure and the at least one reference structure. The similarity can be identified in the respective similarity value can be assigned thereto for the entire molecular structure and reference structure and/or parts thereof. In other words, fingerprints of the molecular structure and the reference structure can be compared to each other in order to identify their similarities or certain similarities of sections thereof. This may further help in providing high similarity and/or property prediction accuracy with high computational and analytical efficiency.

According to a possible embodiment of an assessment method, the step of comparing involves calculating a similarity score of the molecular structure and the at least one reference structure. Any similarity score in general can be applied, in particular a similarity score according to Tanimoto/Jaccard. The similarity score may be based on the similarity value and again may be applied to the entire molecular structure and reference structure and/or parts thereof. This may further help in providing high similarity and/or property prediction accuracy with high computational and analytical efficiency.

According to a possible embodiment of an assessment method, the steps of obtaining the molecular structure and/or of the at least one reference structure involves providing a latent representation of the chemical substance and/or of the at least one reference substance, respectively. The latent representation can be created, generated and/or derived by means of a substance analyzer. The latent representation may comprise nodes representing atoms of the molecular structure and/or the at least one reference structure, as well as edges representing bonds between the atoms. The bonds can be interpreted as chemical connections between atoms of a molecule. Thereby, an efficient and reliable way of assessing new, i.e., non-catalogued or unlabeled, chemical substances may be provided. This can help in providing high similarity and/or property prediction accuracy with a desired degree of flexibility and adaptability, while maintaining a preferably high degree of computational and analytical efficiency.

According to a possible embodiment of an assessment method, the step of comparing involves comparing the latent representation of the molecular structure to the latent representation of the at least one reference structure. Certain property parameters to be identified and/or evaluated may be assigned to the latent representations which can be regarded as models of the structures. A desired degree of abstraction and/or generalization can be applied to the models. This can further help in providing high similarity and/or property prediction accuracy with a desired degree of flexibility and adaptability, while maintaining a preferably high degree of computational and analytical efficiency.

According to a possible embodiment of an assessment method, assessment method further comprises the step of selecting the at least one reference structure of the at least one reference substance from a reference database. The reference database may also hold reference structures of reference substances based on molecule structures which have been analyzed and assessed by means of the assessment method. The database thus helps in facilitating SSL.

According to a possible embodiment of an assessment method, at least one the reference structure and/or the at least one reference substance is selected from a reference cluster containing reference structures and/or reference substances, respectively, being relatively similar (i.e., within a predetermined or defined level of similarity). to each other. The reference database can further contain classifications of reference structures and/or reference substances based on respective reference properties. The clustering and/or the classifications may help in facilitating any selection processes necessary for identifying reference structures, reference substances, and/or reference parameters. This may further help in providing high similarity and/or property prediction accuracy with a high degree of computational and analytical efficiency.

According to a possible embodiment of an assessment method, the step of comparing and/or the step of deriving involves predicting the at least one property parameter by means of a trained prediction algorithm comprising at least one trainable parameter. The trainable parameter may be continuously and/or intermittently trained and adjusted to meet a desired level of similarity and/or property prediction accuracy. Thereby, computational, and analytical efficiency may be further improved.

According to a possible embodiment of an assessment method, the assessment method further comprises the step of training the prediction algorithm by adjusting the trainable parameter through an assessment evaluation of a quality of the prediction of a similarity parameter and/or the at least one property parameter based on a comparison of the at least one reference substance with another reference substance having at least one predetermined similarity score and/or reference parameter, respectively, that serves as a target parameter for the predicted similarity parameter and/or property parameter, respectively. The other words, a predefined similarity parameter and/or at least one property parameter may be used as a target parameter in order to evaluate the quality, i.e., accuracy, of the predicted similarity parameter and/or property parameter, respectively. This helps in further facilitating SSL.

According to a possible embodiment of an assessment method, during a training phase, in particular, a first training phase, the step of comparing the molecular structure to the at least one reference structure is being trained. The first training phase aims at training the assessment of similarities between molecular structures. The training can be based on LSSL, in that a loss function involving a loss L is used in SSL. The loss should be low for all molecular that appears to be compared to each other which is supposed to be having a low similarity value or score and thus may be regarded as rather different to each other. Thereby, in particular a similarity assessment may be trained in a targeted and highly efficient manner.

According to a possible embodiment of an assessment method, during a further training phase, in particular a second training phase, deriving the at least one property parameter from the comparison based on the at least one reference parameter is being trained. The second training phase may follow the first training phase and can be interpreted as a learning phase aiming at assigning properties to the trained assessment of similarities which has been acquired in the first training phase. An N-value score obtained as an output from the first training phase can be used as an input for the second training phase. The N-value score can be a point in a vector space of similarities. Thereby, in particular a property parameter assessment may be trained in a targeted and highly efficient manner.

Consequently, the assessment evaluation may be performed in two steps: A first phase (training phase) may involve using an SSL-loss (LSSL) with similarities for predictions for molecule similarities. A second phase (learning phase) may involve using a loss function, such as a cross entropy loss, indicating a relatively high loss value if a predicted property parameter is relatively different to a target parameter, and indicating a relatively low loss value if a predicted property parameter is relatively similar to a target parameter. Thereby, for example, a semi-supervised learning SSL with a respective loss function LSSL may be applied. A corresponding artificial intelligence (AI)/machine learning (ML) algorithm for comparing the molecular structure to the at least one reference structure and/or for deriving the at least one property parameter from the comparison based on the at least one reference parameter may be implemented.

### BRIEF DESCRIPTION OF THE DRAWINGS

The subject matter will hereinafter be described in conjunction with the following drawing figures, wherein like numerals denote like elements, and wherein:

FIG. 1 is a schematic presentation of an assessment arrangement for a computer assisted assessment of at least one property parameter of a chemical substance.

FIG. 2 is a schematic illustration of a molecular structure of a chemical substance in chemical nomenclature.

FIG. 3 is a schematic illustration of the molecular structure of the chemical substance X shown in FIG. 2 as a latent representation.

FIG. 4 is a schematic diagram illustrating supervised learning step.

FIG. 5 is a schematic diagram illustrating a downstream learning step.

FIG. 6 is a schematic illustration of a number of molecular structures arranged in a virtual three-dimensional space according to their respective similarities.

FIG. 7 is a schematic block diagram illustrating steps of a first training phase.

FIG. 8 is a schematic block diagram illustrating steps of a second training phase.

FIG. 9 is a schematic block diagram of an application phase.

FIG. 10 is a schematic illustration of the chemical substance X shown in FIG. 2 as another latent representation.

FIG. 11 is a schematic illustration of a different chemical substance as a latent representation

FIG. 12 is a schematic illustration of a different chemical substance as a latent representation which has been derived from the chemical substance X shown in FIG. 10 by augmentation.

### DETAILED DESCRIPTION OF THE PREFERRED EMBODIMENTS

The following detailed description is merely exemplary in nature and is not intended to limit the invention and uses of the invention. Furthermore, there is no intention to be bound by any theory presented in the preceding background or the following detailed description. The representations and illustrations in the drawings are schematic and not to scale. Like numerals denote like elements. A greater understanding of the described subject matter may be obtained through a review of the illustrations together with a review of the detailed description that follows.

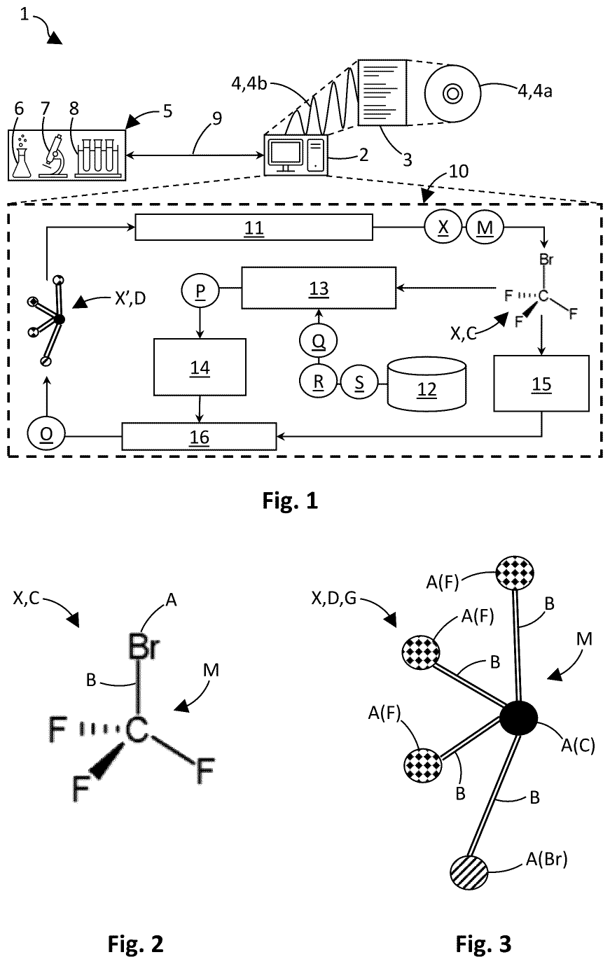

FIG. 1 shows a schematic representation of an assessment arrangement 1 for a computer assisted assessment of at least one property parameter P of a chemical substance X. The assessment arrangement 1 comprises a computing device 2 for implementing an assessment method and/or training method by means of a respective assessment program 3 and/or computer-readable data carrier 4. The computing device 2 is configured to execute the assessment program 3. The computer-readable data carrier 4 has stored thereon the computer assessment program 3 and may take the form of a computer-readable medium 4a and/or data carrier signal 4b.

Furthermore, the assessment arrangement 1 may comprise a laboratory system 5 which may include a reactor device 6, a physical analysis device 7, and/or a chemical analysis device 8. The reactor device 6 may be provided in the form of any kind of chemical reactor for synthesizing and/or decomposing the chemical substance X. The physical analysis device 7 may provide any means for a physical analysis of the chemical substance X and thereby obtaining a respective physical property parameter P_{P} as a form of the property parameter P. The chemical analysis device 8 may provide any means for a chemical analysis of the chemical substance X and thereby obtaining respective chemical property parameter P_{C} as a form of the property parameter P.

The computing device 2 and the laboratory system 5 can be connected to each other via a transmission line 9 for transmitting energy and/or information, such as computational data, or alike, between the computing device 2 and the laboratory system 5. When carrying out the assessment program 3, the computing device 2 provides an assessment and development module 10 for managing, handling, processing and/or visualizing and thus operating all data used in connection with the assessment method and/or training method as described herein. The assessment and development module 10 can be provided with data from the laboratory system 5 and can provide data thereto for developing chemical substances X. Such data may comprise optimization parameters O for optimizing property parameters P by altering the chemical substances X according to respective optimization needs.

The assessment and development module 10 may comprise a computational chemistry engine 11, a database 12, a prediction engine 13, an impact approximation unit 14, a performance approximation unit 15, and a design generator 16. In operation, the computational chemistry engine 11 can provide or suggest a chemical representation C, such as a chemical nomenclature, of the chemical substance X, and can analyze the chemical substance X and provide a molecular structure M and latent representation D thereof. The database 12 may provide data regarding a reference substance S having a reference structure Q and reference parameters R. The data may be provided along with respective chemical representations C and latent representations L.

The prediction engine 13 performs the prediction of the property parameter P by deriving the property partner P from a comparison between the reference substance A with reference parameter R the and the chemical substance X. While the impact approximation unit 14 can approximate an impact of the chemical X on any other substance based on the property parameter P, such as potential impacts on the environment, organisms, technical systems, etc. The performance approximation unit 15 can approximate a certain performance of the chemical substance X, such as its half-life period, decomposition behavior, temperature resistance, pressure resistance, chemical energy, weight, density, or alike. Based on respective impact data and performance data obtained from the impact approximation unit 14, and the performance approximation unit 15, the design generator 16 can suppose the optimization parameter O, for example by suggesting a certain latent representation of an alternative and/or altered chemical substance X′ which can be fed back to the computational chemistry engine 11 for further analysis, alteration, recombination, etc.

FIG. 2 shows a schematic illustration of a molecular structure M of the chemical substance X in chemical representation C. The molecular structure M comprises respective atoms A and bonds B therebetween. In the present example, the chemical representation C of Bromotrifluoromethane consisting of a carbon atom in the center and three fluor and one bromine atom connected to the carbon atom via respective covalent bonds B is illustrated.

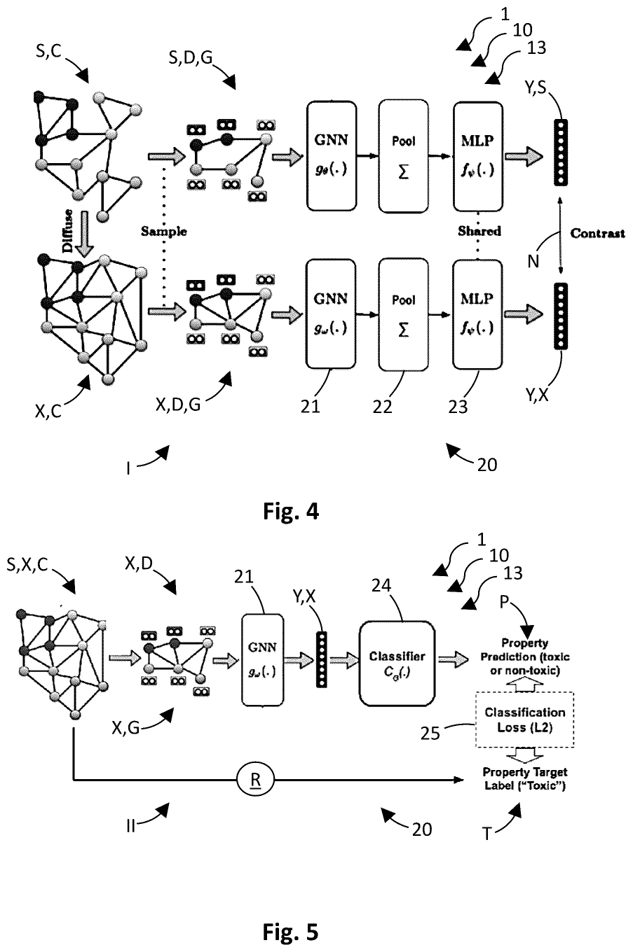

FIG. 3 shows a schematic illustration of the molecular structure M of the chemical substance X shown in FIG. 2 as a latent representation L. Here, the atoms A are represented by nodes and the bonds B therebetween are represented by graphs forming a graphic representation G based on the latent representation L providing respective values embedded in the graphic representation G. Such representation can be used for graph neural networks (GNN) which may be implemented in the assessment and development module 10 and all components thereof (see FIG. 4).

FIG. 4 shows a schematic diagram illustrating supervised learning step the first training phase I which may be implemented as an artificial intelligence (AI) algorithm 20 of the assessment and development module 10, in particular the prediction engine 13 thereof. The AI algorithm 20 can comprise a GNN 21, pooling node 22, a multilayer perceptron (MLP) 23. Chemical representations C of the reference substance S and the chemical substance X can be transformed into their respective latent representations D and graphic representations G which are then each fed to the GNN 21. The output of the GNN 21 is provided to the pooling node 22. The output of the pooling node 22 is provided to the MLP 23. The MLP 23 provides an output value Y for the reference substance S and the chemical substance X which may be compared to each other to identify the contrast therebetween enabling to assess similarity through a similarity score N.

FIG. 5 shows a schematic diagram illustrating a second learning phase II downstream of the first learning phase I. Here, the chemical representation C of the chemical substance X which at the same time can be a reference substance S is transformed into the respective the output value Y is fed into its respective latent representation D and thus the graphic representation G. The graphic representation is fed into the GNN 1 which provides another output value Y. The output value Y is provided to a classifier 24 of the AI algorithm 20. The classifier 24 predicts at least one property parameter P of the chemical substance X.

A discriminator 25 which can also be implemented as a part of the AI algorithm 20 can use respective reference parameter R of the reference substance S previously known. Based on the reference parameter R, the discriminator 25 can derive a target parameter T to be compared to the at least one property parameter P by the discriminator 25. Based on any deviation of the at least one property parameter P and the target parameter T identified by the discriminator 25, the discriminator 25 can issue a loss L, in particular a classification loss which can be fed back to the AI algorithm 20 in order to train and thereby enhance the quality of prediction of the at least one property parameter P.

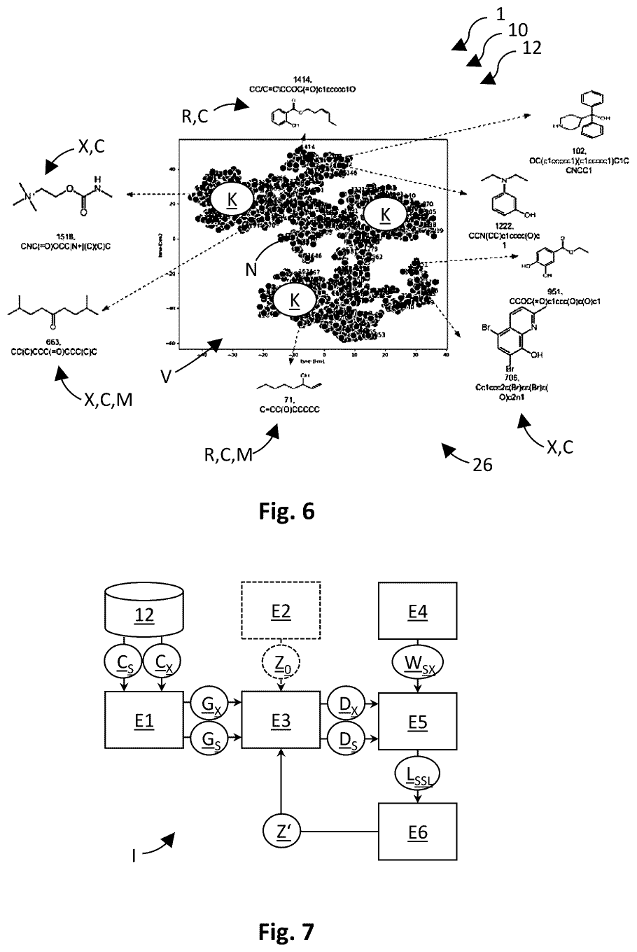

FIG. 6 shows a schematic illustration of a number of molecular structures M of chemical substances X and/or reference substances R arranged in a virtual three-dimensional space V according to their respective similarities which can be expressed by the similarity values N by a graphic engine 26 of the assessment and development module 10. The data for generating the virtual three-dimensional space V containing the similarity values N for the molecular structures X can be organized in the database 12. There, certain groups of molecular structures X may be grouped as clusters K according to certain similar property parameters P or other similarities, such as chemical classifications, that chemical substances X and/or reference substances S assigned to each cluster share.

FIG. 7 shows a schematic block diagram illustrating steps E of a first training phase I where desired and/or required capabilities of the prediction engine 13 are trained by helping the AI engine to emerge. In a first step E1, chemical representations C, for example in the form of the standard nomenclature of the Simplified Molecular Input Line Entry System (SMILES, see https://archive.epa.gov/med/med_archive_03/web/html/smiles.html) taken from the database 12 are converted to respective graphic representations G. In a step E2, a trainable parameter Z can be initialized, for example by providing the trainable parameter Z as a random value for starting value of the trainable starting parameter Z₀. In a step E3, the latent representations D of the reference substances S and/or chemical substances X and can be provided using the graphic representations G thereof and the trainable parameter Z for encoding as follows:
D_{S}=ENCODE_{Z} (G_{S})
D_{X}=ENCODE_{Z} (G_{X})

In a step E4, a pairwise similarity score W can be calculated, for example as a Tanimoto score, for the respective reference substance S and chemical substance X, e.g., in a value range of 0 to 1, as the specific similarity score W_{XS}. The specific similarity score W_{SX} is then issued for the respective pair of reference substance S and chemical substance X. For calculating the similarity score W, respective chemical features determining the similarity scores W of the respective reference substance S and chemical substance X can be managed and stored in the database 12.

In a step E5, the respective loss L is calculated for all pairs of reference substances S and chemical substances X in the database 12 based on the respective latent representations D_{S}, D_{X}. The loss L is high, if a distance dist(D_{S},D_{X}) between the latent representations D is high, while the similarity score W_{SX} between the reference substance S and the chemical substance X is low, as well as if the distance between the latent representations D is low, while the similarity score between the reference substance as in the chemical substance X is high. The loss L can be calculated through SSL as a sub supervised learning loss L_{SSL} as follows:

$${L}_{SSL} = [ log \frac{\sum exp ( dist ( {E}_{X} , {E}_{Y} ) \cdot similarity\_score ( X , Y ) )}{\sum exp ( dist ( {E}_{X} , {E}_{Y} ) \cdot ( 1 - similarity\_score ( X , Y ) ) )} ]$$

In a step E6, the trainable parameter Z can be optimized by finding an optimized trainable parameter Z′ for all pairs of reference substances S and chemical substances X in the database 12, such that a total loss Lₜₒₜₐₗ(L_{SSL}) for all available pairs of reference substances S and chemical substances X reaches a current minimum. The optimized trainable parameter Z′ can then be fed back to step E3 for performing a respective latent encoding. The optimized trainable parameter Z′ can then be used again as the trainable parameter Z for starting another optimization cycle (Z=Z′). Thereby, training phase I enables to train the prediction engine 13, in particular the AI algorithm 20 thereof, so that it learns to organize molecular structures M that are similar to each other on respective clusters K. The encoding can thus be pushed far for being applicable to chemical substances X having different and possibly previously unknown molecular structures M.

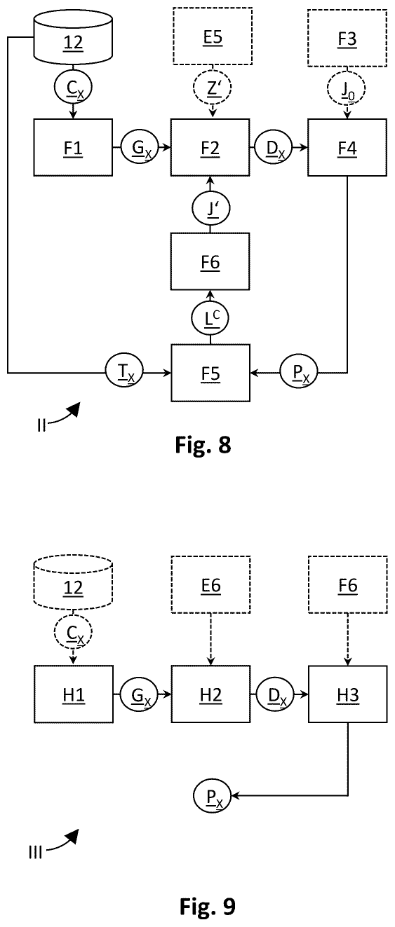

FIG. 8 shows a schematic block diagram illustrating steps F of the second training phase III. In a step F1, a chemical representation C_{X} of the chemical substance X, for example in the form of the standard nomenclature of SMILES taken from the database 12, is converted to respective graphic representations G. In a step F2, the latent representations D of the or chemical substance X and can be provided using the graphic representations G thereof and the optimized trainable parameter Z′ obtained from Step E6 of training phase I for encoding as follows:
D_{X}=ENCODE_{Z′} (G_{X})

In a step F3, another trainable parameter J can be initialized, for example by providing the trainable parameter J as a random value for starting value of the other trainable starting parameter J₀. In a step F4, the property parameter P_{X} of at least one of the chemical substances X is predicted using the respective latent representation D_{X} thereof with the help of the prediction engine 13 as follows:

$${P}_{X} = Property - {Predictor}_{J} ( {D}_{X} )$$

In a step F5, the respective loss L is calculated for the respective predicted property parameter P_{X} and a corresponding target parameter T_{X} obtained from the database 12 for the chemical substance X. The loss L is high, if the predicted property parameter P_{X} and the corresponding target parameter T_{X} are different. The losses low, if the predicted property parameter P_{X} and the corresponding target parameter T_{X} are close to each other, i.e., the same or at least essentially the same. Here, the loss L can be calculated as a cross entropy loss L^{C} for all property parameters P and target parameters T available in the database 12 as follows:
L^{C}=cross_enropy_loss (T_{X},P_{X})

In a step F6, the trainable parameter J can be optimized by finding an optimized trainable parameter J′ for all chemical substances X and property parameters P with respective target parameters T in the database 12, such that a total loss Lₜₒₜₐₗ(L^{C}) is close to a current minimum. The optimized trainable parameter J′ can then be fed back to step F2 for performing a respective latent encoding. The optimized trainable parameter J′ can then be used again as the trainable parameter J for starting another optimization cycle (J=J′). Thereby, training phase II enables to train the prediction engine 13, in particular the AI algorithm 20 thereof, so that it learns to understand property parameters P of molecular structures M based on their respective latent encoding D.

FIG. 9 shows a schematic block diagram illustrating steps H of an application phase III making use of the AI algorithm 20 as it has been trained in training phase I and training phase II. In a step H1, a chemical representation C_{X} of a chemical substance X, for example, a previously unknown chemical substance X, may be obtained in the form of the standard nomenclature of SMILES from the database 12, and is converted to respective graphic representations G. In a step H2, the latent representations D of the or chemical substance X and can be provided using the graphic representations G thereof and the trainable parameter Z obtained from Step E6 of training phase I for encoding by means of the trained AI engine 13 as follows:
D_{X}=ENCODE_{Z} (G_{X})

In a step H3, the latent representations D of the or chemical substance X can be provided the prediction engine 13, including the now trained AI algorithm 20, along with optimized trainable parameter J from step F6. The prediction engine 13 can then predict the property parameter P_{X} of the chemical substance X using the respective latent representation D_{X} with the help of the trained AI algorithm 20 and using respective encoding based on the trainable parameter J as follows:

$${P}_{X} = Property - {Predictor}_{J} ( {D}_{X} )$$

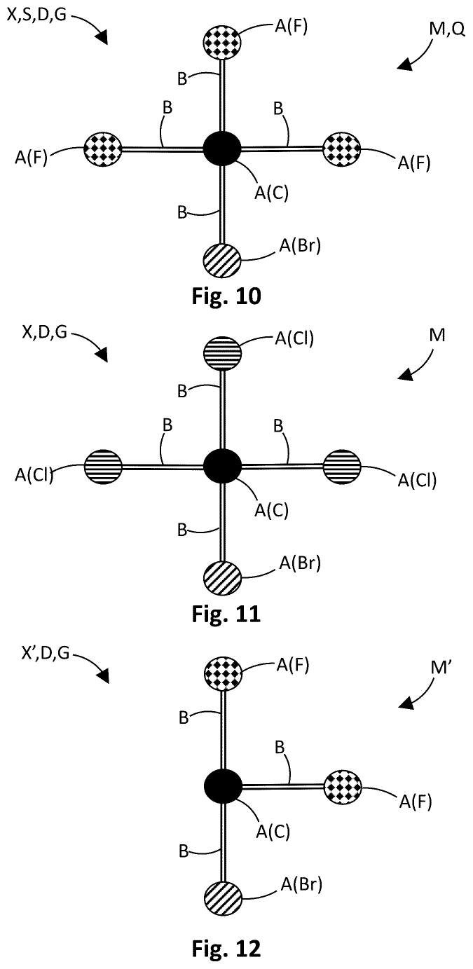

FIG. 10 shows a schematic illustration of the chemical substance X, namely Bromotrifluoromethane (CBrF₃) shown in FIG. 2 as another latent representation D. Through sufficient assessment by means of the assessment and development module 10 through the use of the prediction engine 13, the impact approximation unit 14, and/or the performance approximation unit 15, the chemical substance X has been defined as a reference substance R. As such, the reference substance R can be provided in the database 12 with the respective chemical representation C, for example, as SMILES, in the following form: BrC(F)(F)F.

FIG. 11 shows a schematic illustration of a different chemical substance X as a latent representation D. In the present example, the chemical substance X may be Bromotrichloromethane (CBrCl₃). The at least one property parameter P of the chemical substance X may derived from the respective reference parameter R of the reference substance S by comparing their molecular structure M and reference structure Q, respectively, in the application phase III, the chemical substance X can be provided in the database 12 with the respective chemical representation C, for example, as SMILES, in the following form: ClC(Cl)(Cl)Br.

FIG. 12 shows a schematic illustration of a different chemical substance X′ as a latent representation which has been derived from the chemical substance X shown in FIG. 10 by augmentation. The respective documented graphic representation G can be generated by randomly removing/adding atoms A and bonds B, i.e., nodes and graphs, respectively, from the chemical substance X. This, however, may lead to augmented molecule structures M′ which are unrealistic, i.e., in that the number B of bonds does not match the valency of the atoms A involved. Thus, it may be advantageous, to operate the assessment and development module 10 without augmentation.

The systems and devices described herein may include a controller or a computing device comprising a processing and a memory which has stored therein computer-executable instructions for implementing the processes described herein. The processing unit may comprise any suitable devices configured to cause a series of steps to be performed so as to implement the method such that instructions, when executed by the computing device or other programmable apparatus, may cause the functions/acts/steps specified in the methods described herein to be executed. The processing unit may comprise, for example, any type of general-purpose microprocessor or microcontroller, a digital signal processing (DSP) processor, a central processing unit (CPU), an integrated circuit, a field programmable gate array (FPGA), a reconfigurable processor, other suitably programmed or programmable logic circuits, or any combination thereof.

The memory may be any suitable known or other machine-readable storage medium. The memory may comprise non-transitory computer readable storage medium such as, for example, but not limited to, an electronic, magnetic, optical, electromagnetic, infrared, or semiconductor system, apparatus, or device, or any suitable combination of the foregoing. The memory may include a suitable combination of any type of computer memory that is located either internally or externally to the device such as, for example, random-access memory (RAM), read-only memory (ROM), compact disc read-only memory (CDROM), electro-optical memory, magneto-optical memory, erasable programmable read-only memory (EPROM), and electrically-erasable programmable read-only memory (EEPROM), Ferroelectric RAM (FRAM) or the like. The memory may comprise any storage means (e.g., devices) suitable for retrievably storing the computer-executable instructions executable by processing unit.

The methods and systems described herein may be implemented in a high-level procedural or object-oriented programming or scripting language, or a combination thereof, to communicate with or assist in the operation of the controller or computing device. Alternatively, the methods and systems described herein may be implemented in assembly or machine language. The language may be a compiled or interpreted language. Program code for implementing the methods and systems described herein may be stored on the storage media or the device, for example a ROM, a magnetic disk, an optical disc, a flash drive, or any other suitable storage media or device. The program code may be readable by a general or special-purpose programmable computer for configuring and operating the computer when the storage media or device is read by the computer to perform the procedures described herein.

Computer-executable instructions may be in many forms, including modules, executed by one or more computers or other devices. Generally, modules include routines, programs, objects, components, data structures, etc., that perform particular tasks or implement particular abstract data types. Typically, the functionality of the modules may be combined or distributed as desired in various embodiments.

It will be appreciated that the systems and devices and components thereof may utilize communication through any of various network protocols such as TCP/IP, Ethernet, FTP, HTTP and the like, and/or through various wireless communication technologies such as GSM, CDMA, Wi-Fi, and WiMAX, is and the various computing devices described herein may be configured to communicate using any of these network protocols or technologies.

While at least one exemplary embodiment of the present invention(s) is disclosed herein, it should be understood that modifications, substitutions and alternatives may be apparent to one of ordinary skill in the art and can be made without departing from the scope of this disclosure. This disclosure is intended to cover any adaptations or variations of the exemplary embodiment(s). In addition, in this disclosure, the terms “comprise” or “comprising” do not exclude other elements or steps, the terms “a” or “one” do not exclude a plural number, and the term “or” means either or both. Furthermore, characteristics or steps which have been described may also be used in combination with other characteristics or steps and in any order unless the disclosure or context suggests otherwise. This disclosure hereby incorporates by reference the complete disclosure of any patent or application from which it claims benefit or priority.

### LIST OF REFERENCE SIGNS

1 assessment arrangement
2 computing device
3 assessment program
4 computer-readable data carrier
4a computer-readable medium
4b data carrier signal
5 laboratory system
6 reactor device
7 physical analysis device
8 chemical analysis device
9 transmission line
10 assessment and development module
11 computational chemistry engine
12 database
13 prediction engine
14 impact approximation unit
15 performance approximation unit
16 design generator
20 artificial intelligence algorithm
21 graph neural network
22 pooling node
23 multilayer perceptron
24 classifying unit
25 discriminator
26 graphic engine
I first training phase
II second training phase
III application phase
A Atom
B Bond
C chemical representation/nomenclature
D latent representation
G graphic representation
J trainable parameter
J₀ starting parameter
K cluster
L loss
M molecular structure
N similarity value
O optimization parameter
P property parameter
P_{P} physical property
P_{C} chemical property
Q reference structure
R reference parameter
S reference substance
T target parameter
V virtual space
W similarity score
X chemical substance
Y output value
Z trainable parameter
Z₀ starting parameter
E1 create graphic representations
E2 initialize trainable parameter
E3 calculate latent encoding
E4 calculate similarity score
E5 calculate loss (SSL)
E6 optimize trainable parameter
F1 create graphic representation
F2 calculate latent encoding
F3 initialize trainable parameter
F4 predict property parameter
F5 calculate loss (cross entropy)
F6 optimize trainable parameter
H1 create graphic representation
H2 calculate latent encoding
H3 predict property parameter

Claimed is:

1. A method for computer assisted assessment of at least one property parameter of a chemical substance, comprising the steps of:
obtaining a molecular structure of the chemical substance;
obtaining at least one reference structure of at least one reference substance having at least one reference parameter,
comparing the molecular structure to the at least one reference structure; and
deriving the at least one property parameter from the comparison based on the at least one reference parameter.

2. The method according to claim 1, wherein the step of comparing comprises identifying a similarity between the molecular structure and the at least one reference structure.

3. The method according to claim 1, wherein the step of comparing comprises calculating a similarity score of the molecular structure and the at least one reference structure.

4. The method according to claim 1, wherein obtaining the molecular structure, or obtaining the at least one reference structure, or both comprises providing a latent representation of the chemical substance, a latent representation of the at least one reference substance, or both, respectively.

5. The method according to claim 4, wherein the step of comparing comprises comparing the latent representation of the molecular structure to the latent representation of the at least one reference structure.

6. The method according to claim 1, further comprising the step of:
selecting the at least one reference structure of the at least one reference substance from a reference database.

7. The method according to claim 1, wherein the at least one the reference structure, or the at least one reference substance, or both is selected from a reference cluster containing reference structures, or reference substances, or both, respectively, being relatively similar to each other.

8. The method according to claim 1, wherein the step of comparing, or the step of deriving, or both comprises predicting the at least one property parameter with a trained prediction algorithm comprising at least one trainable parameter.

9. The method according to claim 8, further comprising the step of:
training the trained prediction algorithm by adjusting the trainable parameter through an assessment evaluation of a quality of the prediction of a similarity parameter, or the at least one property parameter, or both based on a comparison of the at least one reference substance with another reference substance having at least one predetermined similarity score, or reference, or both parameter, respectively, that serves as a target parameter for the predicted similarity parameter, or property parameter, or both, respectively.

10. The method according to claim 8, wherein in a first training phase, the step of comparing the molecular structure to the at least one reference structure is being trained.

11. The method according to claim 10, wherein in a second training phase, deriving the at least one property parameter from the comparison based on the at least one reference parameter is being trained.

12. A non-transitory computer-readable data-carrier storing a computer program comprising instructions configured to carry out the method of claim 1.
